(19) United States
(12) Patent Application Publication
(10) Pub. No.: US 2014/0243564 A1
(43) Pub. Date: Aug. 28, 2014

(54) METHOD FOR INHIBITING FOULING IN BASIC WASHING SYSTEMS

(71) Applicant: Baker Hughes Incorporated, Houston, TX (US)

(72) Inventors: Zhenning Gu, Sugar Land, TX (US); Joseph L. Stark, Richmond, TX (US); Roger D. Metzler, Sugar Land, TX (US)

(73) Assignee: Baker Hughes Incorporated, Houston, TX (US)

(21) Appl. No.: 14/272,912

(22) Filed: May 8, 2014

Related U.S. Application Data
(63) Continuation-in-part of application No. 12/062,359, filed on Apr. 3, 2008, now abandoned.

(51) Int. Cl. C07C 7/20; B01D 53/14

(52) U.S. Cl. CPC C07C 7/20; B01D 53/1456
USPC 585/5; 585/3; 585/4; 252/192

(57) ABSTRACT

Fouling in basic washing systems such as caustic scrubbers can be prevented or at least mitigated by treating the liquid washing phase used in a caustic scrubber with an additive having at least one compound selected from the group consisting of oxalyl dihydrazide, a disulfite salt, isopropyl hydroxylamine, chlorobenzhydrazide, aminobenzhydrazide, a thiosulfate salt, 4,4-dimethyl oxazolidine, a hydrosulfite salt, and mixtures thereof, particularly in the case where the liquid washing phase includes components that may undergo an aldol condensation, the products of which may cause fouling solids and/or sludges.

### CROSS-REFERENCE TO RELATED APPLICATION

This application is a continuation-in-part application of U.S. Ser. No. 12/062,359 filed Apr. 3, 2008, incorporated herein by reference in its entirety.

### BACKGROUND

1. Technical Field

The present invention relates to a method for inhibiting fouling in basic washing systems. The present invention particularly relates to the use of additives for inhibiting fouling in basic washing systems.

2. Technical Background

Gas scrubbers are devices used for separating components of a gas admixture. In some embodiments, these devices are used to “purify” gasses or, stated in the alternative, remove undesirable components from a gas stream. For example, primitive scrubbers have been used since the inception of submarine warfare to remove carbon dioxide from the air supply in the submarine.

More recently, gas scrubbers have proven to be essential in many industries. For example, gas scrubbers are used to prevent pollution from the burning of coal during power generation. Gas scrubbers are also used to remove undesired components from crude oil during refining and to remove undesirable components from process gas streams during the production of chemicals, metals and devices such as semiconductors and the like.

### SUMMARY

In one aspect, there is provided a method for the prevention or mitigation of fouling in basic washing systems, where the method comprises treating a liquid washing phase used in a basic washing system having a pH of 9 or greater with an effective amount of an additive to prevent or inhibit fouling, where the additive comprises at least one compound selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof, wherein the liquid washing phase comprises components that may undergo an aldol condensation to cause fouling solids or sludges but for the presence of the additive. The fouling may also have an absence of a chelated polyvalent metal catalyst.

In another aspect, there is provided a liquid washing phase composition used in a basic washing system, where the liquid washing phase composition comprises a caustic liquid that in turn comprises an aqueous solution of a base selected from the group consisting of sodium hydroxide, potassium hydroxide, lithium hydroxide and mixtures thereof; components that may undergo an aldol condensation; and an effective amount of an additive to inhibit or prevent fouling solids or sludges forming from the aldol condensation, but for the presence of the additive, where the additive comprises at least one compound selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof, where the liquid washing phase composition has a pH of 9 or greater, and an absence of a chelated polyvalent metal catalyst.

In yet another aspect, there is provided a method for mitigating the fouling of a caustic scrubber used to wash a gas stream including ethylene where the method comprises treating a liquid washing phase used in the caustic scrubber with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof where the amount of the additive ranges from about 100 to about 50,000 ppm based on the liquid washing phase to prevent or inhibit fouling solids or sludges but for the presence of the additive, where fouling includes an absence of a chelated polyvalent metal catalyst and/or an absence of an aminopolycarboxylic acid chelating agent and/or an absence of a chelated polyvalent metal catalyst; and where the liquid washing phase is a caustic liquid selected from the group consisting of sodium hydroxide, potassium hydroxide, lithium hydroxide and mixtures thereof, and where the liquid washing phase has a pH of 9 or more.

### DETAILED DESCRIPTION

One non-limiting embodiment of the discovery herein includes a method for the prevention or mitigation of fouling in a basic washing system. The most common basic washing systems are caustic scrubbers. For the purposes of the present application, a caustic scrubber is device for removing water soluble and/or acidic or other base reactive components from a fluid stream, often a gas. Also for the purposes of the present application, the term “caustic” is defined broadly to mean a strong base (alkaline) substance including, but not limited to sodium hydroxide, potassium hydroxide, and lithium hydroxide; but also specifically including any compound now known or after discovered useful for extracting a water soluble and/or acidic or other base reactive component from a fluid stream in a fluid scrubber. For example, in one non-restrictive version the basic washing systems useful herein may include an organic amine or a solution including an organic amine as a liquid scrubbing material. However, in another non-limiting embodiment “caustic” is defined a selected from the group consisting of sodium hydroxide, potassium hydroxide, lithium hydroxide, and combinations thereof. It will be appreciated that the fact that a liquid washing phase, which in one non-limiting embodiment is a caustic liquid, does not encompass all liquids that are basic which contain relatively small amounts of an alkali metal hydroxide or alkanolamine, alkyl amine, and/or alkazides to adjust the pH of the liquid. In the caustic liquids used herein, the caustic or basic materials, in the case where alkali metal hydroxide is used in the basic system, the amount of alkali metal hydroxide may be about 20 wt % or less; alternatively about 15 wt % or less; and in another non-limiting embodiment, about 12 wt % or less. In the case where the basic washing system uses amines (alkanolamine, alkyl amine, and/or alkazides), the amount of amine may comprise about 50 wt % independently to about 60 wt % of the liquid, alternatively from about 52 independently to about 58 wt % of the liquid. The basic washing system and/or the liquid washing phase composition may have a pH of 9 or greater; alternatively 9.5 or greater, and in a different non-limiting embodiment of 10 or greater.

Conventional gas scrubbers include a “tower” which is, in essence, a pipe or column, typically including trays or occasionally an inert packing, where a gas stream is contacted with a liquid scrubbing material. In the case of at least one embodiment of the methods and compositions described herein, the liquid scrubbing material is a caustic liquid as defined herein. In some applications, the liquid scrubbing material is passed down through an upward moving gas stream with a packing material serving to increase the mixing of the gas and liquid scrubbing material. In an alternative embodiment, the direction of the gas and fluid passing through the tower may be reversed from that noted in the first embodiment. In still another embodiment, the gas stream and the caustic fluid of the method of the application may move in the same direction.

There are many types of gas scrubbers which are included within the meaning of the term caustic scrubber that are known to be useful. For example, U.S. Pat. No. 6,284,019 to Sorensen, et al., which is fully incorporated herein by reference, discloses a scrubber for removing pollutants from a gas comprising a housing for conducting the gas therethrough, a mechanism for introducing pollutant-reactive filtering agent into the housing, a sump for collecting the filtering agent and having a first volume, and an intermediate sump for collecting the filtering agent and channeling the filtering agent into the sump, the intermediate sump having a second volume that is less than the first volume. Rather than having a tower, this patent discloses the use of a “chamber” which serves the same function as a tower and, for the purposes of the methods and compositions described herein shall be designated as such.

A variety of scrubbers are commercially available. For example, the TRI-MER Corporation markets a variety of scrubbers for use in industry. Exemplary are the so called “cross flow” scrubbers, which come in a variety of configurations. Common to most commercial scrubbers is a “sump” and/or storage tank for scrubbing liquids. These sumps and/or storage tanks may be internal or external to the scrubber cabinets. Any gas scrubber which utilizes a caustic liquid in order to scrub a gas and is known to be useful to those of ordinary skill in the art of using gas scrubbers may be used with embodiments of the method of the disclosure.

With caustic scrubbers, the caustic liquid in the scrubber is sometimes consumed with use, especially when the liquid scrubbing material is an inorganic base such as sodium hydroxide. As a consequence, in such embodiments, the liquid scrubbing material may be renewed either continuously or intermittently. Ideally, it would be desirable in the art that the caustic liquid scrubbing material be renewed only as it is consumed, that is as a consequence of the loss of alkalinity due to reaction of the caustic liquid scrubbing material with acidic, or base reactive compounds in the fluid being scrubbed. Unfortunately, the scrubber may require a more frequent renewal of the caustic fluid, if not an actual shut down and clean out, due to the phenomena of fouling.

While caustic scrubbers are a very common embodiment of basic washing systems, there are other types of basic washing systems in use. Other examples of basic washing systems include scrubbers that use alkanolamines, (such as methyl ethylamine (MEA), diethyl amine (DEA), methyl diethylamine (MDEA), and amine diisopropanol (ADIP)), hindered amines, and alkazide as the liquid washing materials. Still other types of basic washing systems include water wash columns such as those used to refine crude butadiene in butadiene manufacturing plants. Some washing systems are liquid/liquid washing systems where both the washing materials and the stream being washed are liquids and at least some embodiments of the methods and compositions described herein may be used with these applications as well.

For the purposes of the application, fouling, in relation to a basic washing system, occurs when chemical reactions occur in the caustic liquid scrubbing material resulting in solids (or sludges) and/or substantial increases in the viscosity of the caustic liquid scrubbing material. As defined herein, “fouling” is the clogging, blocking, choking or otherwise obstruction of the basic washing system with solids formed by aldol condensation to the point that flow is prevented or inhibited to a problematic extent, meaning preventing the basic washing system from operating in the way in which it was intended. In one non-limiting embodiment, “fouling” includes an absence of consumption of a chelating agent or chelant. In another non-limiting embodiment, the methods and compositions herein may have an absence of an aminopolycarboxylic acid chelating agent and/or an absence of a chelated polyvalent metal catalyst.

While not wishing to be bound by any theory, it is nevertheless believed that when fluids including unsaturated compounds are washed; at least some fouling is the result of aldol polymerizations. In an aldol polymerization, often referred to in the art as an aldol condensation, two molecules, each one having an aldehyde or ketone group react to form a single molecule having a hydroxyl and a carbonyl group. Non-restrictive embodiments of the methods described herein are particularly useful for scrubbing gas streams having components that may undergo an aldol condensation. For example, in one embodiment, the method of the disclosure is used to wash gas including ethylene using a caustic scrubber.

Refineries and chemical plants are among those industries most likely to have fluid streams and especially gas streams which include either acidic or base-reacting gases, or both, that are undesirable and also include aldehyde and/or ketones. Embodiments of the method of the application may be useful in applications where, except for causing fouling, the aldehyde and/or ketone component of a fluid stream is not otherwise undesirable. Embodiments of the methods described herein are also useful in applications where it is desirable to remove an aldehyde or ketone component of a gas stream.

In one other non-limiting embodiment, it is noted that failure to scrub or adequately scrub a gas stream that has a tendency to foul scrubbers is often undesirable in industry. Fouling in scrubbers can be the cause of increased maintenance costs and lost productivity where products are either made at a slower rate because of poor scrubber efficiency or the products produced are out of specification. As an example of the latter, consider a gas stream which may have a CO₂ and/or H₂S specification. Failure to meet the specification may require that the product gas be sent through a second scrubber or refused by a consumer. Either of these can cause production costs to rise with a consequential loss of profits.

Scrubber failures due to fouling may also have safety and environmental consequences. While fouling may be most noticeable in the tower of a scrubber, it may also occur in the sump, holding tank, and in any other pipe, vessel, or other portion of a scrubber where caustic liquid scrubbing material has sufficient residence time to allow for the dropping of suspended solids or the forming of a film or coating onto the walls of the exposed portions of the scrubber.

In some embodiments of the method of the disclosure, a scrubber is treated with an additive. The additive includes at least one member of the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof. In one non-limiting embodiment, the additive does not include alkaline thiosulfate salts or has an absence of thiosulfate salts. In a different non-restrictive version, the additive includes at least one member of the group consisting of oxalyl dihydrazide, chlorobenzhydrazide, aminobenzhydrazide, a disulfite salt, isopropyl hydroxylamine (IPHA), 4,4-dimethyl oxazolidine, a hydrosulfite salt, and mixtures thereof; and in a different non-limiting version the additive does not contain sulfur. For the purposes of this application, salts of these compounds that may form salts are included within the scope of the disclosure even if not specifically specified. For example, even though isopropyl hydroxylamine hydrochloride is not specifically listed, an application where this amine hydrochloride was added to a caustic solution, and thus converted back to an amine, would be within the scope of the disclosure and the claims of this application.

The cations for the salts listed herein may be any cation which would result in a caustic solution soluble salt. For example, sodium thiosulfate and potassium thiosulfate may be used as additives with at least some embodiments of the methods described herein. In one non-restrictive embodiment, the additives include at least one member of the group consisting of: oxalyl dihydrazide, potassium disulfite, isopropyl hydroxylamine (IPHA), 2-chlorobenzhydrazide, 4-aminobenzhydrazide, sodium thiosulfate, 4,4-dimethyl oxazolidine, sodium hydrosulfite, and mixtures thereof. While, in one embodiment, the additive is a solid which can be added directly or indirectly to a caustic liquid scrubbing material, in other embodiments, the additive is added as a solution or suspension.

The additives of one non-restrictive aspect of the present methods and compositions may be in the form of an aqueous solution, possibly including a dispersant, of oxalyl dihydrazide, chlorobenzhydrazide, aminobenzhydrazide, potassium disulfite, isopropyl hydroxylamine (IPHA), sodium thiosulfate, 4,4-dimethyl oxazolidine, sodium hydrosulfite, and mixtures thereof. When the additives are in the form of a solution, generally the concentration the oxalyl dihydrazide, chlorobenzhydrazide, aminobenzhydrazide, potassium disulfite, isopropyl hydroxylamine (IPHA), sodium thiosulfate, 4,4-dimethyl oxazolidine, sodium hydrosulfite, and mixtures thereof will be from about 0.1 independently to about 50 weight percent. In some embodiments, the concentration will be from about 5 independently to about 30 weight percent and in others, from about 7 independently to about 20 weight percent. The term “independently” as used herein with respect to a range means that any lower threshold may be combined with any upper threshold for the same range type to form a suitable alternative range.

Any dispersant useful for dissolving or suspending one or more of these compounds in water may be used with at least some of the embodiments of the methods. In addition, one of the compounds may be dissolved or suspended in a water soluble or miscible solvent. Exemplary of such materials include, but are not necessarily limited to: mono-ethylene glycol n-hexyl ether (HEXYL CELLOSOLVE® available from Union Carbide); ethylene glycol monobutyl ether (BUTYL CELLOSOLVE®); di- and tri-propylene glycol derivatives of propyl and butyl alcohol, which are available from Arco Chemical (3801 West Chester Pike, Newtown Square, Pa. 19073) and Dow Chemical (1691 N. Swede Road, Midland, Mich.) under the trade names ARCOSOLV® and DOWANOL®; mono-propylene glycol mono-propyl ether; di-propylene glycol mono-propyl ether; mono-propylene glycol mono-butyl ether, di-propylene glycol mono-propyl ether, di-propylene glycol mono-butyl ether; tri-propylene glycol mono-butyl ether; ethylene glycol mono-butyl ether; di-ethylene glycol mono-butyl ether, ethylene glycol mono-hexyl ether; di-ethylene glycol mono-hexyl ether; 3-methoxy-3-methyl-butanol; and mixtures thereof. In regard to these solvents, “butyl” includes normal butyl, isobutyl and tertiary butyl groups. Mono-propylene glycol and mono-propylene glycol mono-butyl ether can be used and are available under the trade names DOWANOL DPnP® and DOWANOL DPnB®. Di-propylene glycol mono-t-butyl ether is commercially available from Arco Chemical under the tradename ARCOSOLV PTB®. In some instances, it might be preferred to use combinations of these solvents, such as HEXYL CELLOSOLVE with BUTYL CELLOSOLVE, or DOWANOL PnB with 3-methoxy-3-methyl-butanol.

The additives described herein are desirably fed to basic washing systems such as scrubbers at an effective concentration. Those of ordinary skill in the art of running such units are well versed in determining the effective concentration of additives to use in their equipment. Such concentrations are dictated, in the case of gas scrubbers for example, by the operational conditions of the scrubbers including the makeup of the gas stream, feed rates, and operating temperatures. Generally, the additives will be present at a level in the caustic solutions in the scrubbers such that the concentration of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 100 independently to about 50,000 ppm. In other embodiments the concentration is from about 1000 independently to about 5000 ppm.

The additives may be desirably added to a liquid feed stream into a basic washing system such as, for example, a caustic scrubber. The additive may be added directly to the caustic scrubber or aspirated into a gas feed stream. The additives may also be introduced into a basic washing system using any other method known to be useful for introducing an additive to a scrubber.

In addition to the additive components already described, the additives used herein may include other compounds known to be useful in basic washing systems such as dispersants, defoamers, and the like. Any compound that does not have an undesirable interaction with the additive's ability to prevent fouling may be used with at least some embodiment of the methods and compositions described herein.

### EXAMPLES

The following examples are provided to illustrate the present invention. The examples are not intended to limit the scope of the present invention and they should not be so interpreted. Amounts are in weight parts or weight percentages unless otherwise indicated.

### Example 1

A blank was prepared by admixing spent caustic bottoms (pre-filtered to remove existent solid particles) dosed with 6000 ppm of vinyl acetate and with 5% oil. Samples 1-1 through 1-6 were prepared by admixing the blank material with the additive shown below in Table 1. The dosed caustic sample was heated at 80° C. for sixteen hours. Once the heating period was complete, the sample was allowed to cool to ambient. The caustic was filtered through a 1.0-1.5 μm-7.0 cm glass fiber filter. The filter paper was dried and weighed. The weight gain is reported as potential polymer/fouling formation. Inhibitor efficacy was measured in comparison to sample blank.

TABLE 1

| | | Additive | Potential | % |
|---|---|---|---|---|
| Sample | Additive | Dosage | Sediment | Fouling |
| # | dosed | (ppm) | (mg/100 ml) | inhibition |
| BLANK | — | — | 245.5 to 315.0** | — |
| 1-1 | Oxalyl | 8200 | 89.0 | 66.9 |
| | Dihydrazide | | | |
| 1-2 | Potassium | 4650 | 112.7 | 64.2 |
| | Disulfite | | | |
| 1-3 | IPHA | 5250 | 116.7 | 52.5 |
| 1-4* | NaBH₄ | 660 | 208.1 | 33.6 |
| 1-5 | Sodium | 11000 | 235.5 | 25.1 |
| | Thiosulfate | | | |
| 1-6 | 4, 4-Dimethyl | 9400 | 241.8 | 10.2 |
| | Oxazolidine | | | |

*Comparative Example.
**The blank material appeared to degrade with time. Blanks were rerun with each test and % fouling was determined using a blank run near in time with the designated sample.

### Example 2

10 ml of 2% NaOH was added to 20 ml glass vials. One vial was used as a blank and left untreated. The remaining vials were treated with sufficient vinyl acetate to result in a concentration of 1100 ppm. One of the vials was sealed used as a control. The remaining vials were treated with additives as shown below in Table 2. All four vials were capped and shaken well before settling in an oven at 55° C. overnight. Table 2 shows the results of observation.

TABLE 2

| Sample | Inhibitor | Result |
|---|---|---|
| Blank | None | Clear, colorless |
| Control | None | Hazy, yellow colored |
| 2-1 | 2-chlorobenzoic hydrazide | Clear, very light |
| | (3400 ppm) | yellow colored |
| 2-2 | 4-aminobenzoic hydrazide | Clear, ivory colored |
| | (3400 ppm) | |

### Example 3

A control sample was prepared by dosing 6000 ppm of vinyl acetate into 2% NaOH solution. Samples were prepared by dosing into a control sample the additives shown below in Table 3. The dosed caustic sample was heated at 80° C. for sixteen hours. Once the heating period was complete, the sample was allowed to cool to ambient. The caustic was filtered through a 1.0-1.5 micrometer glass fiber filter that was 7.0 cm in diameter. The filter paper was dried and weighed. The weight gain is reported as potential polymer/fouling formation. Inhibitor efficacy was measured in comparison to the control sample.

TABLE 3

| | | Additive | Potential | % |
|---|---|---|---|---|
| Sample | Additive | Dosage | Sediment | Fouling |
| # | dosed | (ppm) | (mg/100 ml) | Inhibition |
| Control | — | — | 112.0-118.4 | — |
| 3-1 | 25% Sodium | 3000 | 54.1 | 54.3 |
| | hydrosulfite in | | | |
| | Caustic Solution | | | |
| 3-2 | 18% Sodium | 3750 | 25.4 | 78.5 |
| | hydrosulfite in | | | |
| | Caustic Solution | | | |

Many modifications may be made in the methods and apparatus of this invention without departing from the spirit and scope thereof that are defined only in the appended claims. For example, the basic washing systems, liquid washing phases, caustic liquids, additives, fouling solids or sludges, proportions, dosages, process sequences, and the like may be different from those mentioned and used here, and the additives may be used in different proportions from those mentioned and used here, and still be within the methods and compositions described.

The words “comprising” and “comprises” as used throughout the claims is interpreted as “including but not limited to”.

The present invention may suitably comprise, consist or consist essentially of the elements disclosed and may be practiced in the absence of an element not disclosed. For instance, there may be provided a method for the prevention or mitigation of fouling in basic washing systems, the method consisting essentially of or consisting of treating a liquid washing phase used in the basic washing system with an effective amount of an additive to prevent or inhibit fouling, where the additive comprises a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof, wherein the liquid washing phase comprises components that may undergo an aldol condensation to cause fouling solids or sludges but for the presence of the additive

In another non-limiting embodiment there may be alternatively provided a method for mitigating the fouling of a caustic scrubber used to wash a gas including ethylene, where the method consists essentially of or consists of treating a liquid washing phase used in the caustic scrubber with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof; where the amount of the additive ranges from about 100 to about 50,000 ppm based on the liquid washing phase to prevent or inhibit fouling solids or sludges but for the presence of the additive, where fouling includes an absence of an aminopolycarboxylic acid chelating agent; where the liquid washing phase is a caustic liquid selected from the group consisting of sodium hydroxide, potassium hydroxide, lithium hydroxide and mixtures thereof.

In a different, non-restrictive version there may be provided a liquid washing phase composition used in a basic washing system, the liquid washing phase composition consisting essentially of or consisting of a caustic liquid comprises an aqueous solution of a base selected from the group consisting of sodium hydroxide, potassium hydroxide, lithium hydroxide and mixtures thereof; components that may undergo an aldol condensation; an effective amount of an additive to inhibit or prevent fouling solids or sludges forming from the aldol condensation, but for the presence of the additive, where the additive comprises a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof.

What is claimed is:

1. A method for the prevention or mitigation of fouling in basic washing systems, the method comprising treating a liquid washing phase used in the basic washing system having a pH of 9 or greater with an effective amount of an additive to prevent or inhibit fouling, where the additive comprises a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof, wherein the liquid washing phase comprises components that may undergo an aldol condensation to cause fouling solids or sludges but for the presence of the additive; where the fouling has an absence of a chelated polyvalent metal catalyst.

2. The method of claim 1 where the fouling includes an absence of an aminopolycarboxylic acid chelating agent.

3. The method of claim 1 where the basic washing system is a scrubber.

4. The method of claim 3 where the scrubber is a gas scrubber.

5. The method of claim 4 where the gas scrubber is used to wash a gas comprising ethylene.

6. The method of claim 1 where the liquid washing phase is a caustic liquid.

7. The method of claim 6, where the caustic liquid comprises an aqueous solution of a member selected from the group consisting of sodium hydroxide, potassium hydroxide, lithium hydroxide and mixtures thereof.

8. The method of claim 7 where the caustic liquid comprises an aqueous solution of sodium hydroxide.

9. The method of claim 1 where the additive includes a compound selected from the group consisting of potassium disulfite, sodium thiosulfate, sodium hydrosulfite, and mixtures thereof.

10. The method of claim 1 where the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 0.1 to about 50 weight percent.

11. The method of claim 1 where the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 5 to about 30 weight percent.

12. The method of claim 1 where the additive comprises an aqueous solution and wherein the concentration of the oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof is from about 7 to about 20 weight percent.

13. The method of claim 1 where the effective amount of the additive selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof in the liquid washing phase is a concentration of from about 100 to about 50,000 ppm.

14. The method of claim 1 where the effective amount of the additive selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof in the liquid washing phase is a concentration of from about 1,000 to about 5,000 ppm.

15. The method of claim 1 where the additive is selected from the group consisting of oxalyl dihydrazide, chlorobenzhydrazide, aminobenzhydrazide, isopropyl hydroxylamine (IPHA), 4,4-dimethyl oxazolidine, and mixtures thereof.

16. A method for mitigating the fouling of a caustic scrubber used to wash a gas including ethylene, the method comprising:
treating a liquid washing phase used in the caustic scrubber with an additive comprising a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof; where the amount of the additive ranges from about 100 to about 50,000 ppm based on the liquid washing phase to prevent or inhibit fouling solids or sludges but for the presence of the additive, where fouling includes; where the fouling has an absence of a chelated polyvalent metal catalyst;
where the liquid washing phase is a caustic liquid selected from the group consisting of sodium hydroxide, potassium hydroxide, lithium hydroxide and mixtures thereof.

17. The method of claim 16 where the additive is selected from the group consisting of oxalyl dihydrazide, chlorobenzhydrazide, aminobenzhydrazide, isopropyl hydroxylamine (IPHA), 4,4-dimethyl oxazolidine, and mixtures thereof

18. A liquid washing phase composition used in a basic washing system, the liquid washing phase composition comprising:
a caustic liquid comprises an aqueous solution of a base selected from the group consisting of sodium hydroxide, potassium hydroxide, lithium hydroxide and mixtures thereof;
components that may undergo an aldol condensation;
an effective amount of an additive to inhibit or prevent fouling solids or sludges forming from the aldol condensation, but for the presence of the additive, where the additive comprises a member selected from the group consisting of oxalyl dihydrazide, chlorobenzhydrazide, aminobenzhydrazide, a disulfite salt, isopropyl hydroxylamine (IPHA), a thiosulfate salt, 4,4-dimethyl oxazolidine, a hydrosulfite salt, and mixtures thereof;
where the liquid washing phase composition has a pH of 9 or greater, and has an absence of a chelated polyvalent metal catalyst

19. The composition of claim 18 where the fouling inhibited or prevented includes an absence of an aminopolycarboxylic acid chelating agent.

20. The composition of claim 19 where the effective amount of the additive is a concentration of from about 100 to about 50,000 ppm based on the liquid washing phase.
